(19) United States
(12) Patent Application Publication
(10) Pub. No.: US 2024/0209033 A1
(43) Pub. Date: Jun. 27, 2024

(54) MAMMALIAN CELL LINE AND METHOD FOR PRODUCING SOLUBLE E2 RECOMBINANT ANTIGEN OF CLASSICAL SWINE FEVER VIRUS USING THE SAME AND APPLICATION THEREOF

(71) Applicant: National Pingtung University Of Science And Technology, Pingtung (TW)

(72) Inventors: Hso-Chi Chaung, Pingtung (TW); Wen-Bin Chung, Pingtung (TW); Chi-Chih Chen, Pingtung (TW)

(73) Assignee: National Pingtung University Of Science And Technology, Pingtung (TW)

(21) Appl. No.: 18/485,754

(22) Filed: Oct. 12, 2023

(30) Foreign Application Priority Data
Dec. 26, 2022 (TW) 111149970

(51) Int. Cl. C07K 14/005; A61K 39/00; C12N 5/071; C12R 1/91

(52) U.S. Cl. CPC C07K 14/005; C12N 5/0682; A61K 39/00; C12N 2770/24322; C12N 2770/24334; C12R 2001/91

(57) ABSTRACT

The present disclosure relates to a mammalian cell line and a method for producing soluble E2 recombinant antigen of classical swine fever virus using the same. A mammalian cell expression system with different cell passage numbers can stably express a large amount of soluble CSFV-E2 recombinant protein, effectively reducing the production cost of CSFV-E2 recombinant protein, and then applied to the production of CSFV-E2 protein subunit vaccines.

### RELATED APPLICATION

This application claims priority to Taiwan Application Serial Number 111149970, filed Dec. 26, 2022, which is herein incorporated by reference.

### REFERENCE TO THE SEQUENCE LISTING

A sequence listing is being submitted herein as xml format with the name “SP-5910-US_SEQ_LIST”, created on Sep. 11, 2023, with a file size of 4,799 bytes.

### BACKGROUND

### Field of Invention

The present disclosure relates to a mammalian cell line, in particular to a mammalian cell line stably expressing soluble E2 recombinant antigen of classical swine fever virus in mass and a method for producing the soluble E2 recombinant antigen of classical swine fever virus using the same and application thereof.

### Description of Related Art

Classical swine fever (CSF), also known as classical swine fever, is a highly contagious disease that infects pigs. Generally, high fever, anorexia, diarrhea, neurological symptoms, erythema all over the body, mass death, porcine abortion or giving birth to dead piglets in pigs are highly contagious and highly pathogenic diseases that infect pigs.

After CSF was discovered for the first time in 1903, it became popular all over the world, including Asia, Africa, Europe, Central and South America etc. Once an epidemic broke out, it often caused major economic losses in the pig industry, and even affected the global economy. Countries where CSF is endemic must use routine vaccination to prevent and control the spread of CSF. When used properly, vaccination can effectively limit the spread of CSF, prevent disease outbreaks and establish protective immunity in uninfected pig herds.

The pathogen of CSF is classical swine fever virus (CSFV), which belongs to a Pestivirus of the Flavivirdae. CSFV genome is a single-stranded positive-sense RNA with a total length of about 12.3 kb, which will be translated into 3,898 amino acids to form polyprotein, and then processed by protease into four structural proteins (C, Erns, E1 and E2) and eight non-structural viral proteins (Npro, p7, NS2, NS3, NS4A, NS4B, NS5A and NS5B), in which E2, Erns and NS3 can induce the host to produce antibody responses.

Currently, there are two main commercialized CSF vaccines: CSF modified live vaccine (MLV) and CSFV envelope protein E2 subunit vaccine. Although the CSF MLV vaccine is cheap and effective, it is impossible to distinguish the immune response of pigs generated from MLV vaccination or natural infection by serum antibody detection. After pigs are inoculated with the CSF MLV vaccine, CSFV in the environment can continue to infect animals, and the immune response of animals after natural infection can still cause a certain degree of low virus load in animals, resulting in persistence of CSFV in the environment and in the host. In order to completely eradicate the source of infection of the virus, it is necessary to distinguish whether the pig that response in the body is a vaccine-immunized pig or a naturally infected pig with the virus in the animal farm to perform an eradication plan. Therefore, a diagnostic reagent is required to distinguish between the two types of the immune responses.

Although commercially available insect cells can produce E2 recombinant antigen, the cost remains high. Therefore, it is an urgent issue to develop low-cost E2 subunit vaccines. In view of this, there is an urgent need to develop a method for producing low-cost E2 recombinant antigen to solve various issues in the prior art.

### SUMMARY

Accordingly, one aspect of the present disclosure is to provide a mammalian cell line stably expressing soluble E2 recombinant antigen of classical swine fever virus.

Another aspect of the present disclosure is to provide a method for producing the soluble E2 recombinant protein of classical swine fever virus, which cultures the aforementioned mammalian cell line to increase the production of the soluble E2 recombinant antigen.

A further aspect of the present disclosure is to provide the soluble E2 recombinant antigen of classical swine fever virus, which is produced by the aforementioned mammalian cell line.

A further aspect of the present disclosure is to provide a porcine subunit vaccine composition, which uses the aforementioned soluble E2 recombinant antigen of classical swine fever virus as an active ingredient.

According to the above aspect of the present disclosure, the mammalian cell line stably expressing the soluble E2 recombinant antigen of classical swine fever virus is provided, which is Chinese hamster ovary cell line CCC_E2_5E12 deposited in German Collection of Microorganisms and Cell Cultures GmbH (Deutsche Sammlung von Mikroorganismen und Zellkulturen, DSMZ) (Inhoffenstraße 7B, 38124 Braunschweig, Science Campus Braunschweig-Süd, GERMANY) on May 17, 2023 and assigned accession number DSM ACC3376.

In the foregoing embodiments, an amino acid sequence of the soluble E2 recombinant protein can be, for example, as shown in SEQ ID NO:1.

In the foregoing embodiments, a protein concentration of the soluble E2 recombinant protein can be, for example, at least 800 mg/L cell culture fluid. In some examples, a protein concentration of the soluble E2 recombinant protein can be, for example, at least 850 mg/L cell culture fluid. In other examples, a protein concentration of the soluble E2 recombinant protein can be, for example, in a range of from 850 mg/L cell culture fluid to 950 mg/L cell culture fluid.

In the foregoing embodiments, the E2 recombinant protein can be, for example, encoded by a nucleic acid sequence shown in SEQ ID NO:2.

According to the other aspect of the present disclosure, a method for producing the soluble E2 recombinant protein of classical swine fever virus is provided, which cultures the aforementioned mammalian cell line to obtain the soluble E2 recombinant antigen.

According to the further aspect of the present disclosure, the soluble E2 recombinant antigen of classical swine fever virus is provided, which is produced by the aforementioned mammalian cell line, in which an amino acid sequence of the soluble E2 recombinant protein can be, for example, as shown in SEQ ID NO:1.

According to the further aspect of the present disclosure, a porcine subunit vaccine composition is provided, which uses the aforementioned soluble E2 recombinant antigen of classical swine fever virus as an active ingredient, and a number of administrations for the porcine subunit vaccine composition to be effective to a test subject is one.

In the foregoing embodiments, an effective dose of the soluble E2 recombinant protein of classical swine fever virus can be, for example, in a range of from 25 μg/mL to 50 μg/mL.

Applying the mammalian cell line and the method for producing the soluble E2 recombinant antigen of classical swine fever virus of the present disclosure, it can utilize a mammalian cell expression system with different cell passage numbers to greatly increase the production of the soluble E2 recombinant antigen of classical swine fever virus, which can effectively reduce the production cost of the soluble E2 recombinant antigen of classical swine fever virus, and then applied to the production of CSFV-E2 protein subunit vaccines.

It can be understood that the foregoing general description and the following detailed description are only examples, and are intended to provide further explanations to the claimed invention.

### BRIEF DESCRIPTION OF THE DRAWINGS

The disclosure can be more fully understood by reading the following detailed description of the embodiment, with reference made to the accompanying drawings as follows.

FIG. 1A and FIG. 1B respectively show a colloidal image of SDS-PAGE (FIG. 1A) and an image of Western blot (FIG. 1B) of a culture medium of transfected CHO cells according to an embodiment of the present disclosure.

FIG. 2A to FIG. 2B respectively show a colloid image of SDS-PAGE (FIG. 2A) and an image of Western blot (FIG. 2B) of E2 recombinant protein expressed by CHO cell line CCC_E2_5E12 according to an embodiment of the present disclosure.

FIG. 3A to FIG. 3B respectively show a colloid image of SDS-PAGE (FIG. 3A) and an image of Western blot (FIG. 3B) of CHO cell line CCC_E2_5E12 with different cell passage numbers expressing E2 recombinant protein in cell fluid before purification according to an embodiment of the present disclosure.

FIG. 4A to FIG. 4B respectively show a colloid image of SDS-PAGE (FIG. 4A) and an image of Western blot (FIG. 4B) of CHO cell line CCC_E2_5E12 with different cell passage numbers expressing E2 recombinant protein in cell fluid after purification according to an embodiment of the present disclosure.

FIG. 5 is a bar graph showing a protein concentration of purified E2 recombinant protein in cell fluid of CHO cell line CCC_E2_5E12 with different cell passage numbers according to an embodiment of the present disclosure.

### DETAILED DESCRIPTION

If the definition or usage of a term in the cited document is inconsistent with or contrary to the definition of the term here, the definition of the term here applies instead of the definition of the term in the cited document. Moreover, unless otherwise defined by context, the singular term can include plural, and the plural term can include singular.

As mentioned above, the present disclosure provides a mammalian cell line and a method for producing soluble E2 recombinant antigen of classical swine fever virus using the same, which can mass-produce the soluble E2 recombinant antigen of classical swine fever virus by using a mammalian cell expression system.

The terms “recombinant protein”, “recombinant antigen”, “protein”, “peptide” and “exogenous polypeptide” referred to herein are interchangeable and refer to polymer of amino acids, usually bonded by peptide bonds or disulfide bonds. The term “peptide” can also be used in amino acids and polymer thereof that one or more amino acid residues are naturally occurring, or amino acid polymer corresponding to analogs or mimetics of naturally occurring amino acids. The term “peptide” further includes modified amino acid polymer, e.g., glycoprotein with carbohydrate residues, or phosphorylated peptides. The peptides, exogenous polypeptide and protein can be produced by liquid-phase synthesis, solid-phase synthesis, or genetic engineering, recombinant cells, prokaryotic expression systems, and eukaryotic expression systems. In one embodiment, the recombinant antigen referred to herein is soluble classical swine fever virus E2 (CSFV-E2) recombinant protein produced by the mammalian cell expression system.

The terms “amino acid” and “residue” referred to herein are interchangeable, and when used in combination with peptide, those refer to naturally occurring and synthetic amino acids, amino acid analogs, amino acid mimetics and non-naturally occurring amino acids that are chemically similar to naturally occurring amino acids.

The term “classic swine fever virus (CSFV)” referred to herein, there is no particular limitation on the type of virus strain, such as subgroup 2.1a of CSFV. However, in other embodiments, other virus strains can also be used for CSFV, depending on actual needs.

The term “E2 recombinant protein” referred to herein can be, for example, full-length E2 recombinant protein, such as an amino acid sequence shown in SEQ ID NO:1, or an amino acid sequence encoded by a nucleic acid sequence shown in SEQ ID NO:2, and the amino acid sequence from the N-terminal to the C-terminal is a signal peptide (favorable for secretion of translated E2 protein into a culture medium), a sequence-modified E2 protein (HS2 strain, about 342 a.a.), protease cleavage sites (favorable for cutting a His tag) and a His tag (good for protein purification). The amino acid sequence shown in SEQ ID NO:1 can be obtained by extracting a gene fragment of E2 [equivalent to the 1,267th to the 2,292nd nucleotide; a corresponding E2 amino acid fragment is the 423rd to the 764th amino acid of a complete sequence (GenBank No. AAS20410.1)] based on a gene sequence of 2.1a gene subtype of CSFV (GenBank No. AY526726.1) published by Pan et al. (Archives of Virology, volume 150, pages1101-1119, 2005) and modifying it into a sequence suitable for production by the mammalian cell expression system. In some specific examples, in order to facilitate construction of recombinant plastids, restriction enzyme cleavage sites can be selectively added to both ends of the E2 recombinant gene sequence. The type of the restriction enzyme cleavage site is not particularly limited, depending on the sequence of the plastid to be constructed, such as restriction enzyme cleavage sites of Bam HI and Not I. In addition, in order to facilitate subsequent protein purification, the C-terminal of the E2 recombinant protein can be optionally added with a human rhinovirus 3C protease (HRV 3C protease) cleavage site (about 8 a.a., which is conducive to removal of a His tag) and a His tag (His tag; or polyhistidine), in which the His tag can include but not limited to 6 to 10 histidine residues. Therefore, the E2 recombinant protein obtained above is soluble protein.

In some embodiments, the CSFV-E2 recombinant protein can be produced using known methods or following methods. Firstly, prokaryotic transformed cells are subjected to a protein expression step, in which the prokaryotic transformed cells can include a first recombinant plastid, which contains a recombinant gene of a nucleic acid sequence shown in SEQ ID NO:2, to express the CSFV-E2 recombinant protein of the amino acid sequence shown in SEQ ID NO:1. For the subsequent purification of the recombinant protein, a nucleic acid sequence for encoding a His tag can be selectively added to the 3′ end of the above-mentioned recombinant gene. In some examples, the aforementioned nucleic acid sequence for encoding the His tag can be provided by a commercially available plastid, and designed to be connected to the 3′ end of the aforementioned recombinant gene. The nucleic acid sequence of the His tag is known in the technical field of the present disclosure and will not be further described here.

Next, the first recombinant plastid of the prokaryotic transformed cells is extracted, and the E2 recombinant gene is cut by the restriction enzyme and constructed to a second recombinant plastid suitable for the mammalian cell expression system, and mammalian cells were transfected with the second recombinant plastid, and a large amount of the E2 recombinant protein is expressed and secreted into the cell culture medium, and the soluble E2 recombinant protein was recovered and purified.

In the above embodiments, the type of the mammalian cells is not particularly limited, and can be, for example, a commercially available Chinese hamster ovary cell (CHO cell) line.

In the above embodiments, after the E2 recombinant protein is recovered, the CSFV-E2 recombinant protein can be selectively subjected to a conventional column purification step to obtain purified CSFV-E2 recombinant protein.

Generally, cells of different cell passage numbers of the above-mentioned mammalian cells can stably express and significantly increase production of CSFV soluble E2 recombinant antigen, in which a yield of the CSFV-E2 recombinant protein can be at least 800 mg of the soluble CSFV-E2 recombinant protein obtained from per liter of the cell culture medium, or at least 850 mg, or 850 mg to 950 mg of the soluble CSFV-E2 recombinant protein. It is worth mentioning that the E2 recombinant protein produced by the mammalian cell expression system can be secreted into the cell culture medium, which can effectively increase the yield of the E2 recombinant protein, and its yield is at least 80-fold to 100-fold higher than those of other expression systems (e.g., the expression system of baculovirus-infected insect cells). Moreover, the E2 recombinant protein produced by the mammalian cell expression system has a high proportion of the soluble protein, and at least 95% of the E2 recombinant protein is soluble protein. Therefore, the E2 protein can be recovered and purified using a lower-cost process, thereby greatly reducing the production cost of producing CSF E2 recombinant protein. Furthermore, the proportion of the soluble E2 recombinant protein produced by the mammalian cell expression system is relatively high, which can effectively improve effectiveness of prepared vaccines. In some embodiments, one-time administration of the vaccine made of the soluble E2 recombinant protein can reach 100% protection. In contrast, about 50% to 80% of the E2 recombinant protein produced by other expression systems (e.g., the expression system of the baculovirus-infected insect cells) is insoluble protein, and a yield of soluble protein is low, which cannot be purified. A vaccine made using unpurified E2 recombinant protein is not only less effective, but also uses a higher dose of antigen, so two doses are required (i.e., additional vaccination is required) to achieve 100% protection. In other words, the dosage of the E2 recombinant protein produced by other expression systems (e.g., the expression system of the baculovirus-infected insect cells) is twice the dosage of the soluble E2 recombinant protein produced by the mammalian cell expression system. Moreover, the E2 recombinant protein produced by other expression systems (e.g., the expression system of the baculovirus-infected insect cells) cannot be purified and can contain allergens, and after such prepared vaccines are vaccinated into animals, these can cause side effects (e.g., allergic reactions and even death) in some animals, thereby reducing safety of vaccines.

The above-mentioned soluble CSFV-E2 recombinant protein can form a bivalent or multivalent antigen with other antigenic protein, and only a single dose vaccination can provide effective immune protection. The term “single dose vaccination” mentioned here refers to a number of administrations of a porcine subunit vaccine composition to a test subject to be effective is one without need for booster immunization, and without side effects, and thus can effectively simplify a vaccination process.

In some embodiments, an effective dose of the CSFV-E2 recombinant protein of the porcine subunit vaccine composition can be, for example, 2 mL per dose, and each dose contains 25 μg/mL to 50 μg/mL. In some specific examples, the effective dose of the CSFV-E2 recombinant protein can be, for example, 25 μg/mL.

It should be understood that the following specific recombinant protein sequences, specific formulations, specific dosages, specific detection methods, viewpoints, illustrations and embodiments are for illustration only, and not as limitations of the present disclosure. The principal features of the present disclosure can be employed in various embodiments without departing from the spirit and scope of the present disclosure. Therefore, those skilled in the art of the present disclosure can easily determine the essential technical features of the present disclosure, and make various changes and modifications to the present disclosure to apply to different purposes and conditions without departing from the spirit and scope of the present disclosure.

### Embodiment: Preparation of CSFV-E2 Recombinant Protein

### 1. Construction of CSFV-E2 Recombinant Gene

The amino acid sequence of the CSFV-E2 recombinant protein is shown in SEQ ID NO:1, which was obtained by extracting the gene fragment of E2 [equivalent to the 1,267th to the 2,292nd nucleotide; the corresponding E2 amino acid fragment was the 423rd to the 764th amino acid of the complete sequence (GenBank No. AAS20410.1)] based on the gene sequence of the 2.1a gene subtype of CSFV (GenBank No. AY526726.1) published by Pan (2005) et al., and synthesizing and modifying into the sequence suitable for expression through the mammalian expression system by Mission Biotech Co., Ltd., and designing the restriction enzyme cleavage sites of Bam HI and Not I at both ends, and adding the signal peptide to the N-terminal (protein-tyrosine phosphatase, PTP; a total of 21 a.a., which was conducive to the secretion of the translated E2 protein into the culture medium), and adding the HRV 3C protease cleavage site (a total of 8 a.a., which was conducive to the removal of the His tag) and the His tag (His tag, a total of 8 a.a, which was beneficial for protein purification) to the C-terminal, and cloning into a pcDNA3.4 expression vector and named as PTP-E2, and its nucleic acid sequence is shown in SEQ ID NO:2.

### 2. Screening of Cell Line Stably Expressing CSFV-E2 Recombinant Protein

A commercially available mammalian cell expression system was used in the embodiment, such as a commercially available CHO cell expression system (ExpiCHO™ expression system, product model A29129, Thermo Fisher Scientific Inc. Waltham, MA, USA, and imported by KIM FOREST ENTERPRISE co., ltd). The E2 recombinant protein was produced according to an operation manual (Pub. No MAN0017764, Rev. 3.0).

At first, a plastid of PTP-E2/pcDNA3.4 was cut into a linear vector using the Sspl restriction enzyme (BioLabs, R0132S), and then transfected into ExpiCHO™-S cells using the ExpiFectamine™ CHO transfection reagent (thermo, A29129). The transfected ExpiCHO™-S cells were firstly cultured in the ExpiCHO™ expression medium (model A2910001, thermo) containing 200 μg/mL of G418 sulfate (Uni-onward Corp., G8168). In addition, the transfected ExpiCHO™-S cells were seeded into a shake flask (125 mL) containing 30 mL of the ExpiCHO™ expression medium at a cell density of 3×10⁵ cells/mL, and 4 g/mL of glucose was added on the days 3, 5 and 7, and the cell culture fluid was collected on the days 0, 3, 5, 7, 10, 12 and 14 to analyze the expression of the E2 recombinant protein.

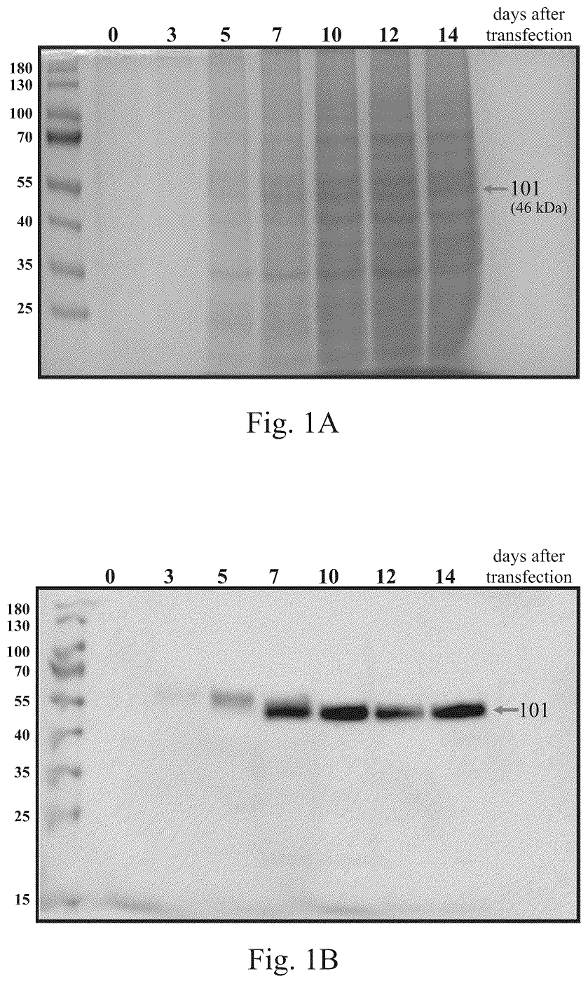

Sodium dodecyl sulfate polyacrylamide gel electrophoresis (SDS-PAGE) and Western blot of protein transfer were used to analyze the recombinant protein described in section 2.1, and results were shown in FIG. 1A (SDS-PAGE: E2) and FIG. 1B [Western blot: E2, detected by anti-E2 antibody (product name: WH303, model: RAE0826, Genhot Laboratories Inc.)].

Please refer to FIG. 1A and FIG. 1B, which respectively showed the colloidal image of SDS-PAGE (FIG. 1A) and the image of Western blot (FIG. 1B) of the culture medium of the transfected CHO cells according to an embodiment of the present disclosure.

The results in FIG. 1A showed that the E2 recombinant protein was successfully expressed in the supernatant, as shown by an arrow 101 (E2 monomer, 46 kDa) in FIG. 1A. The results in FIG. 1B showed that the recombinant protein expressed by the transfected CHO cells was confirmed to be the E2 recombinant protein (its sequence is shown in SEQ ID NO:1).

Next, a single cell was isolated by limiting dilution cloning, and then expanded to obtain a cell line stably expressing the recombinant CSFV-E2 protein.

It is stated that after the cells were serially diluted to a cell concentration of 1,000 cells/mL, the cell solution of 0.1 mL was added to the expression culture solution of 39.9 mL, and it was added to a 96-well plate with a volume of about 200 μL per hole and then cultured at 37° C. and a carbon dioxide concentration of 8%. It was cultured until the day 17, and the culture solution containing cells in each well was transferred to a 24-well plate, and 300 μL of the expression culture solution was added to each well, and each well contained about 0.5 mL of the culture solution, and cultured at 37° C. and a carbon dioxide concentration of 8%. Finally, a cell bank of 235 cell clones was obtained, and the cell line with the highest recombinant protein expression (i.e., the mammalian cell line stably expressing the soluble CSFV-E2 recombinant antigen) was selected according to the protein yield evaluation and deposited in German Collection of Microorganisms and Cell Cultures GmbH (Deutsche Sammlung von Mikroorganismen und Zellkulturen, DSMZ) (Inhoffenstraße 7B, 38124 Braunschweig, Science Campus Braunschweig-Süd, GERMANY), and the name was Chinese hamster (Cricetulus griseus) ovary cell CCC_E2_5E12, and the deposit date was May 17, 2023, and the accession number was DSM ACC3376, which was confirmed the survival of the cell line on Jun. 7, 2023.

### 3. Expression and Purification of E2 Recombinant Protein

Before transfection, the CHO cell line CCC_E2_5E12 (cell density reached 3×10⁵ cells/mL) and a total of 30 mL of the stable medium (ExpiCHO™ stable production AGT medium, model A3711101, thermo) were cultured at 37° C. and a carbon dioxide concentration of 8%. On the days 3, 5 and 7 of the culture, glucose with a final concentration of 4 g/L was added. On the day 14 of the culture, it was centrifuged at 2,000 g for 15 minutes to separate the cells and remove the supernatant (a part of the culture solution). Afterwards, ammonium sulfate (Sigma, Product No. A4915) was added to the supernatant for precipitation to obtain 20-40% precipitate. Next, the precipitate was redissolved in 100 mL of a buffer A [containing 50 mM Tris and 500 mM sodium chloride (NaCl)], and its pH value was pH 7.5. Next, after the redissolved product was filtered using a 0.45 μm filter membrane, a column purification step was performed, and the culture solution was injected into the Ni-NTA column, and an eluate (containing 50 mM Tris, 500 mM NaCl and 250 mM imidazole) was used to elute the E2 recombinant protein to obtain the E2 recombinant protein with high purity.

### 4. Verification of Performance of Recombinant Protein

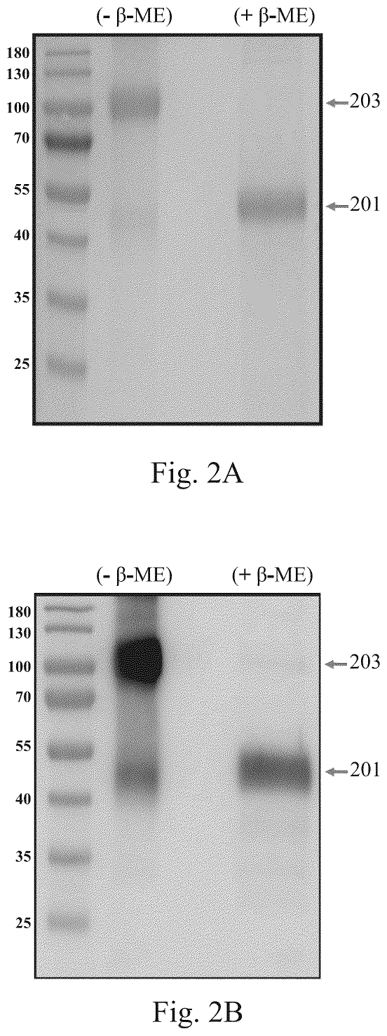

The SDS-PAGE and Western blot of protein transfer were used to analyze the recombinant protein described in section 2.1, and results were shown in in FIG. 2A (SDS-PAGE: E2) and FIG. 2B [Western blot: E2, detected by anti-E2 antibody (product name: WH303, model: RAE0826, Genhot Laboratories Inc.)].

The above-mentioned SDS-PAGE film containing the E2 recombinant protein was transferred to a polyvinylidene difluoride (PVDF) membrane (GE healthcare, Taiwan-Level Biotechnology Inc.) and reacted with 20 ml of a blocking buffer [Tris buffered saline with Tween 20 (TBST) and 5% bovine serum albumin (BSA)] at room temperature (about 25° C.) for 2 hours. Next, the blocking buffer was removed, and the PVDF membrane was reacted with 10 ml of anti-His tag antibody solution [antibody and TBST buffer solution (1×PBS with 0.1% Tween-20) with a dilution factor of 1:1000] at 4° C. for 16 to 18 hours. Afterwards, the anti-His tag antibody solution was removed, and the PVDF membrane was rinsed with 10 mL of TBST buffer solution three times for 20 minutes each time. Next, the PVDF membrane was reacted with the TBST buffer solution of anti-mouse antibody (a dilution factor 1:10000) at room temperature for 1 hour. Afterwards, the TBST buffer solution containing the antibody was removed, and 10 mL of the TBST buffer solution was added to wash the PVDF membrane three times for 20 minutes each time. Next, the PVDF membrane was colored with a coloring agent (ECL stain kit, Taiwan-Level Biotechnology Inc.), and results are shown in FIG. 2B.

Please refer to FIG. 2A and FIG. 2B, which respectively showed the colloid image of SDS-PAGE (FIG. 2A) and the image of Western blot (FIG. 2B) of the CSFV-E2 recombinant protein expressed by the CHO cell line CCC_E2_5E12 according to an embodiment of the present disclosure. FIG. 2A, from left to right, Lane 1 was the protein marker; Lane 2 was purified recombinant CSFV2 E2 treated without β-mercaptoethanol (β-ME) (E2 dimer; −β-ME); Lane 3 was purified CSFV2 E2 recombinant protein treated with β-ME, and its molecular weight was 46 kDa.

The results in FIG. 2A showed that the supernatant of the CHO cell line CCC_E2_5E12 could indeed purify the CSFV-E2 recombinant protein, as shown by an arrow 201 (E2 monomer, a molecular weight of 46 kDa; treated with β-mercaptoethanol, +B-ME) and an arrow 203 (E2 dimer, a molecular weight of 92 kDa; treated without β-ME, −β-ME) in FIG. 2A. The results in FIG. 2B showed that the protein purified from the supernatant of the CHO cell line CCC_E2_5E12 was confirmed to be the CSFV E2 recombinant protein (its sequence is shown in SEQ ID NO:1).

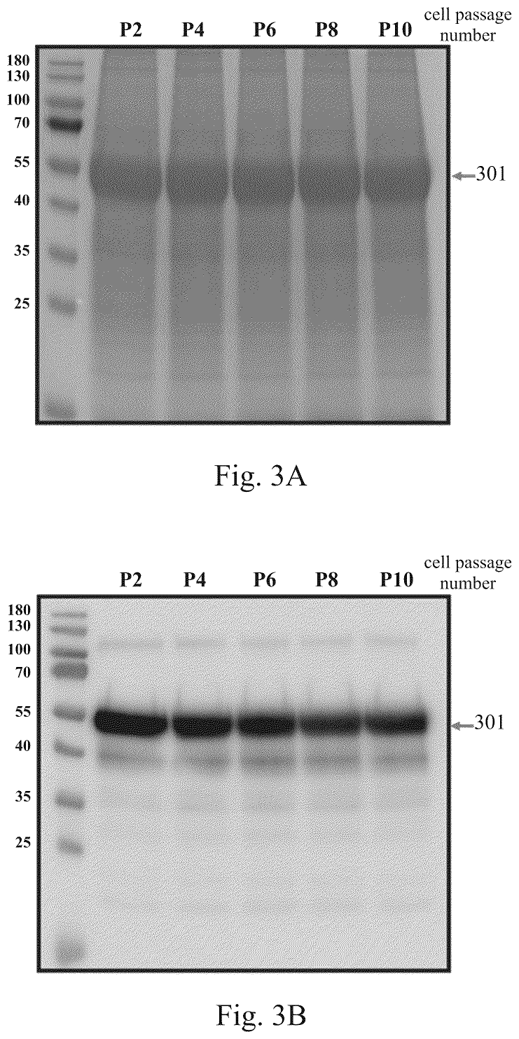

In order to confirm whether expression levels of the CSFV E2 recombinant protein of the CHO cell line CCC_E2_5E12 of different cell passage numbers were stable, the SDS-PAGE and Western blot of protein transfer were carried out on the cell passage numbers 2, 4, 6, 8 and 10 of the CHO cell line CCC_E2_5E12 obtained from the culture solution, and results are shown in FIG. 3A and FIG. 3B, respectively.

Please refer to FIG. 3A to FIG. 3B, which respectively showed the colloid image of SDS-PAGE (FIG. 3A) and the image of Western blot (FIG. 3B) of the CHO cell line CCC_E2_5E12 with different cell passage numbers expressing the E2 recombinant protein in the cell fluid before purification according to an embodiment of the present disclosure.

The results of FIG. 3A and FIG. 3B showed that the supernatant of the CHO cell line CCC_E2_5E12 of the different cell passage numbers before purification, the E2 recombinant protein could indeed be stably expressed in the supernatant, as shown in band signals indicated by arrows 301 in FIG. 3A and FIG. 3B, which were similar.

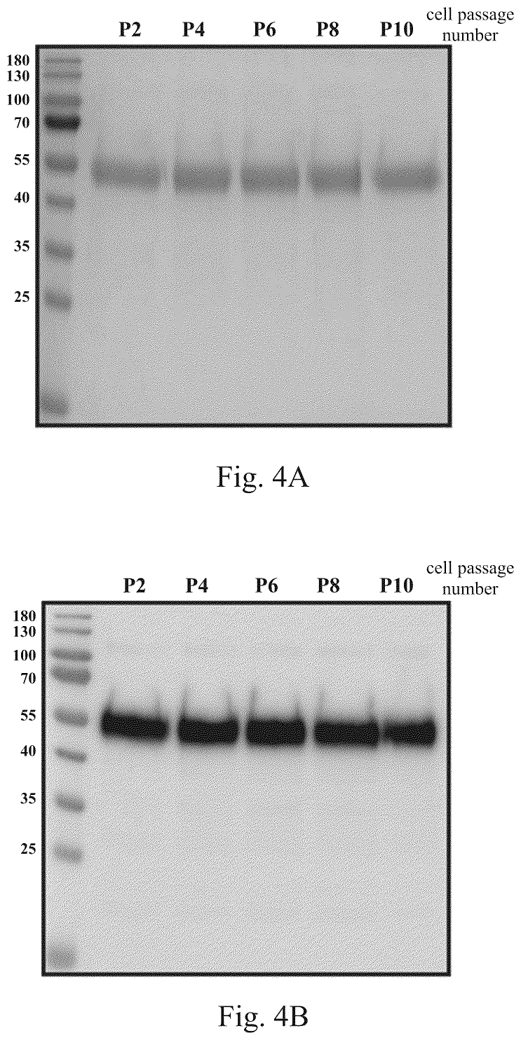

Please refer to FIG. 4A to FIG. 4B, which respectively showed the colloid image of SDS-PAGE (FIG. 4A) and the image of Western blot (FIG. 4B) of the CHO cell line CCC_E2_5E12 with different cell passage numbers expressing the E2 recombinant protein in the cell fluid after purification according to an embodiment of the present disclosure.

The results of FIG. 4A and FIG. 4B showed that the supernatant of the CHO cell line CCC_E2_5E12 of the different cell passage numbers after purification, the E2 recombinant protein could indeed be stably expressed in the supernatant, as shown in band signals in FIG. 4A and FIG. 4B, which are similar.

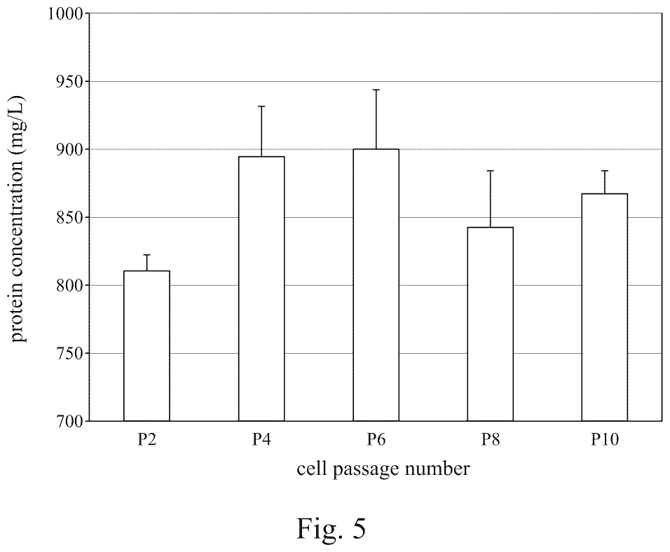

In addition, commercially available electrophoresis colloid densitometry method was used to detect concentrations of the purified E2 recombinant protein of the CHO cell line CCC_E2_5E12 with different cell passage numbers, and results were shown in FIG. 5.

Please refer to FIG. 5, which was a bar graph showing the protein concentration of the purified E2 recombinant protein in the cell fluid of the CHO cell line CCC_E2_5E12 with different cell passage numbers according to an embodiment of the present disclosure. The results in FIG. 5 showed that after the supernatants of the CHO cell line CCC_E2_5E12 with different cell passage numbers were purified, there was no statistically significant difference (T-test statistical analysis, P>0.05) in the expression of the E2 recombinant protein of the CHO cell line with different cell passage numbers, in which an average protein concentration of the E2 recombinant protein was at least 800 mg/L cell culture fluid, preferably at least 850 mg/L cell culture fluid, more preferably 850 mg/L cell culture fluid to 950 mg/L cell culture fluid.

The aforementioned embodiments confirmed that the mammalian cell expression system of different cell passage numbers could stably express and greatly increase the production of the soluble CSFV-E2 recombinant antigen, and the production cost of producing the CSFV-E2 protein was effectively reduced, and then applied to the production of CSFV-E2 protein subunit vaccines.

In summary, the specific amino acid sequences, specific cell lines, specific producing methods or specific evaluation methods are only used to illustrate the mammalian cell line and its use in the production of the soluble CSFV-E2 recombinant antigen. However, those skilled in the art of the present disclosure should understand that without departing from the spirit and the scope of the present disclosure, other amino acid sequences, other production methods or other evaluation methods can also be used for the mammalian cell line and the method for producing the soluble CSFV-E2 recombinant antigen, and not limited to the above.

According to the foregoing embodiments, advantages of the mammalian cell line of the present disclosure and the method for producing the soluble CSFV-E2 recombinant antigen lies in the use of the mammalian cell expression systems with different cell passage numbers, which can greatly increase the production of the soluble CSFV-E2 antigen, and the production cost of producing CSFV-E2 protein is effectively reduced, and then applied to production of CSFV-E2 protein subunit vaccines.

While the present disclosure has been disclosed above with several specific embodiments, other embodiments are possible. Therefore, the spirit and scope of the appended claims of the present disclosure should not be limited to the description of the embodiments contained herein.

What is claimed is:

1. A mammalian cell line stably expressing soluble E2 recombinant antigen of classical swine fever virus, which is Chinese hamster ovary cell line CCC_E2_5E12 deposited in German Collection of Microorganisms and Cell Cultures GmbH (Deutsche Sammlung von Mikroorganismen und Zellkulturen, DSMZ) (Inhoffenstraße 7B, 38124 Braunschweig, Science Campus Braunschweig-Süd, GERMANY) on May 17, 2023 and assigned accession number DSM ACC3376.

2. The mammalian cell line stably expressing the soluble E2 recombinant antigen of the classical swine fever virus of claim 1, wherein an amino acid sequence of the soluble E2 recombinant protein is shown in SEQ ID NO:1.

3. The mammalian cell line stably expressing the soluble E2 recombinant antigen of the classical swine fever virus of claim 1, wherein a protein concentration of the soluble E2 recombinant protein is at least 800 mg/L cell culture fluid.

4. The mammalian cell line stably expressing the soluble E2 recombinant antigen of the classical swine fever virus of claim 1, wherein a protein concentration of the soluble E2 recombinant protein is at least 850 mg/L cell culture fluid.

5. The mammalian cell line stably expressing the soluble E2 recombinant antigen of the classical swine fever virus of claim 1, wherein a protein concentration of the soluble E2 recombinant protein is in a range of from 850 mg/L cell culture fluid to 950 mg/L cell culture fluid.

6. The mammalian cell line stably expressing the soluble E2 recombinant antigen of the classical swine fever virus of claim 1, wherein the E2 recombinant protein is encoded by a nucleic acid sequence shown in SEQ ID NO:2.

7. A method for producing soluble E2 recombinant protein of classical swine fever virus, which cultures the mammalian cell line of claim 1 to obtain the soluble E2 recombinant antigen.

8. The method for producing the soluble E2 recombinant protein of the classical swine fever virus of claim 7, wherein a protein concentration of the soluble E2 recombinant protein is at least 800 mg/L cell culture fluid.

9. A porcine subunit vaccine composition, comprising a soluble E2 recombinant antigen of classical swine fever virus as an active ingredient, wherein the soluble E2 recombinant antigen is produced by the mammalian cell line of claim 1, and an amino acid sequence of the soluble E2 recombinant protein is shown in SEQ ID NO:1.

10. The porcine subunit vaccine composition of claim 9, wherein the E2 recombinant protein is encoded by a nucleic acid sequence shown in SEQ ID NO:2.

11. The porcine subunit vaccine composition of claim 9, wherein a number of administrations for the porcine subunit vaccine composition to be effective to a test subject is one.

12. The porcine subunit vaccine composition of claim 11, wherein an effective dose of the soluble E2 recombinant protein of the classical swine fever virus is in a range of from 25 μg/mL to 50 μg/mL.


SEQUENCE LISTING (2 sequences)

SEQ ID NO: 1 - protein, 371 aa, synthetic construct
  source 1..371: note = synthetic construct
MKVLRGQIVQGIIWLLLVTGAQGRLSCKEDHRYAISSTNEIGPLGAEGLTTTWKEYNHGLQLDDGTVRAICIAGSFRVTALNVVSRRYLASLHKRALPTSVIFELLFDGTSPAIEEMGDDFGFGLCPFDTTPVVKGKYNTTLLNGSAFYLVCPIGWTGVIECTAVSPTTLRTEVVKTFKREKPFPHRADCVTTIVEKEDLFHCKLGGNWTCVKGNPVTYTGGQVKQCRWCGFDFKEPDGLPHYPIGKCILANETGYRIVDSTDCNRDGVVVSTEGEHECLIGNTTVKVYALDGRLAPMPCRPKEIISSAGPVRKTSCTFNYTKTLRNKYYEPRDSYFQQYMLKGEYQYWFDLDVTGHHADYFAEGHHHHHH

SEQ ID NO: 2 - DNA, 1116 bp, synthetic construct
  source 1..1116: mol_type = other DNA; note = synthetic construct
atgaaagtattgagaggacagatcgtgcaaggcataatatggctgttgctggtgactggggcacaagggcggttgtcctgtaaggaagaccacaggtatgcaatatcatcaaccaatgagataggaccgctaggggctgaaggtctcaccactacctggaaagaatacaatcatggtttgcagctggatgacgggactgtcagggctatttgcattgcagggtcctttagagtcacggcacttaatgtggttagcaggaggtacctggcgtcattacataagagggctttgcccacttcagtgatatttgaactcctatttgatgggaccagcccagcaattgaggagatgggagatgactttggatttggactgtgcccttttgacacgactcctgtggtcaaagggaagtacaataccactttattaaacggcagcgcattctatctagtctgcccaatagggtggacgggtgtcatagagtgcacggcagtaagccccacaaccttgagaacagaagtggtgaagaccttcaagagagagaagcctttcccgcacagagcggattgcgtgaccactatagtagaaaaagaagacctgttccactgtaagttggggggcaactggacatgtgttaaaggcaacccggtgacctacacgggggggcaagtaaaacaatgcaggtggtgtggttttgacttcaaggagcctgatgggctcccacactaccccataggcaagtgcatcttagcaaatgagacgggttacaggatagtggactccacagactgcaacagagatggcgttgtcgttagcactgaaggagaacatgagtgcttgattggcaacactactgtcaaagtatacgcattggatggaagactggcccctatgccgtgtagacccaaagaaatcatctctagcgcgggacctgtaaggaaaacttcctgcactttcaattacacaaagactctaagaaacaagtactatgagcccagggacagctatttccagcaatatatgcttaagggcgagtaccaatactggtttgatctggacgtgactggccaccatgcagactacttcgccgaaggtcatcaccatcaccatcactga